(19) United States
(12) Patent Application Publication
(10) Pub. No.: US 2024/0350102 A1
(43) Pub. Date: Oct. 24, 2024

(54) METHOD AND SYSTEM FOR CREATING A SIMULATED FOOT POSITION

(71) Applicant: Orthios Innovations, LLC, North Bergen, NJ (US)

(72) Inventors: Richard P. Adelson, North Bergen, NJ (US); James P. Duncan, Hernando, MS (US); David R. Kuenstler, San Antonio, TX (US); Carl C. Gauger, Tucson, AZ (US); David J. Pancratz, Helotes, TX (US)

(73) Assignee: Orthios Innovations, LLC, North Bergen, NJ (US)

(21) Appl. No.: 18/605,131

(22) Filed: Mar. 14, 2024

Related U.S. Application Data
(60) Provisional application No. 63/588,331, filed on Oct. 6, 2023.
(60) Provisional application No. 63/497,709, filed on Apr. 22, 2023.

(51) Int. Cl. A61B 6/04; A61B 6/50; A61B 90/00; A61G 13/12

(52) U.S. Cl. CPC A61B 6/0421; A61B 6/505; A61G 13/125; A61G 13/129; A61B 2090/3966; A61G 2210/50

(57) ABSTRACT

A system for creating a simulated foot position, with or without weightbearing, includes a base configured to support a foot thereon and a footpad securable with the base in an upright position. The footpad in the upright position receives the foot thereagainst such that the footpad maintains the foot in a simulated foot position. The footpad pivotally secures with the base such that the footpad moves between the upright position and a stowed position. The footpad further pivotally secures with the base such that the upright position of the footpad includes multiple upright positions having different angles that provide the footpad with multiple simulated foot positions.

### BACKGROUND OF THE INVENTION

### 1. Field of the Invention

The present invention relates to a system capable of creating an effective and reproducible simulated foot position as well as ankle position of a (1) sedated patient, (2) an awake but non-ambulatory patient, or (3) an ambulatory but resting patient. The invention also includes a method of use for creating this simulated foot position.

### 2. Description of the Related Art

During foot and ankle surgery, or in a clinical setting for non-ambulatory or resting patients, orthopedic surgeons frequently desire to see the normal positioning of bones and joints of a patient's foot. A patient resting or sedated on a bed does not have their foot in a normal, weightbearing position. Instead, the foot of a resting or sedated patient would have the toes extended downward and outward, with bones oriented differently than while walking. In this position, the surgeon cannot visualize the effectiveness of surgery or walking anatomy. To view a patient's foot in a normal standing or walking position, the physician desires to create a simulated weightbearing position for the foot, and then hold the foot in this position while performing imaging studies. Imaging can include x-rays (digital such as C-arm or film-based), CT scans, or MRI images. The physician would like this simulated weightbearing position to be reproducible, so that the images have the same orientation and relative bone positioning as the surgery progresses. In addition, as the patient heals post-operatively or in a clinical setting, the physician can continue to create a simulated weightbearing position for the foot with the same orientation and relative positioning.

Full view simulated weightbearing images assist the surgeon to assure that any fixation that is being placed in the foot is in the proper position in the foot's normal standing or walking position. In addition, simulated weightbearing images provide an accurate assessment of the surgical correction that took place, and will be predictive of the series of post-operative x-rays that will be taken as the patient heals.

There are no systems available for creating an effective and reproducible simulated weightbearing position for a patient's foot, and that allow hands-free imaging. Existing solutions involve manually using a metallic or plastic object to press against the patient's foot, with different amounts of force and from different angles, creating a non-reproducible and unpredictable outcome. In addition, existing solutions involve operating room or healthcare providers to be in the field of the imaging equipment, creating unnecessary radiation or electromagnetic field exposure.

Accordingly, a system is described herein for creating reproducible, repeatable simulated foot positions such as simulated weightbearing positions of a foot in a resting or sedated patient, while allowing healthcare staff to remain clear of the imaging field of view. Various configurations of the system and methods of use for the system are also presented. The system allows fast deployment and stowage of a footpad that can apply pressure during imaging.

### SUMMARY OF THE INVENTION

The invention herein consists of a medical device system, and a method for using the system. The system has several embodiments and components, suitable for different healthcare settings. The system may be provided to a hospital or surgery center sterile, or non-sterile, depending on the needs of the facility. The system may also operate in a clinical office setting.

The system is designed to sit on a hospital bed or surgical table. It can be sterilized, or left non-sterile. It can be carried to the operating room by grasping the handle on the footpad. The system can then be placed on the bed or table, at any location. The system can be prepared for a surgical procedure and imaging by moving the calfplate into the elevated position, if desired. The patient's foot and calf is positioned on the calfplate. The heelcup holds the patient's operative foot steady during all procedures. The footpad can then be moved from the stowed position to the upright position, or an interim position, by the operating room staff. The footpad can then be returned to the stowed position, and this motion between stowed and upright position can be performed as many times as needed during the surgical procedure or imaging study. The patient's foot and leg can remain on the simulated foot positioning system throughout the procedure to make it as convenient as possible for the operating room staff.

A simulated foot position represents any scenario in which a physician or healthcare professional wishes to temporarily position a patient's foot and lower leg in a specific position, such that images can be taken (x-ray, fluoro, MRI, CT, or other), surgical corrections can be performed, a visual assessment can be conducted, or a biomechanical motion can be performed. A simulated foot position can have a negligible force applied to the bottom of the foot, or it can include a substantial force applied to the bottom of the foot, such as when simulating weightbearing. A simulated foot position can also be at various angles, from plantar-flexed (toes away from the head) to dorsiflexed (toes towards the head). Simulated weightbearing positions can be used to simulate the effect of walking, running, dancing, or any other numerous activities of daily life that a physician would find useful in assessing the medical condition and any surgical intervention.

When used in this way, the physician has a platform for performing surgery that holds the foot and lower leg steady, and elevated if desired. Repeatable and reproducible simulated foot position images such as simulated weightbearing images can be taken quickly and as many times as desired by moving the footpad from stowed position to upright position, and back again. Interim positions provide additional options for the surgeon. A handle on the footpad and latches on the calfplate allow the system to be stowed and transported conveniently and easily.

A system for creating a simulated foot position, in accordance with the present invention, includes a base and a footpad securable with the base in an upright position. The base is configured to support the foot thereon, and, in a preferred embodiment, the base includes a base plate and a calfplate securable with the base plate configured to support the foot thereon. The footpad in the upright position receives the foot thereagainst such that the footpad maintains the foot in a simulated foot position. The footpad pivotally secures with the base plate such that the footpad moves between the upright position and a stowed position whereby the footpad inserts between the base plate and the calfplate. The footpad further pivotally secures with the base plate such that the upright position of the footpad includes multiple upright positions having different angles that provide the footpad with multiple simulated foot positions. The footpad includes at least one radiopaque trim piece. The footpad includes first and second strap slots configured to receive a strap therethrough that affixes the foot to the footpad, whereas the base plate includes first and second strap slots configured to receive a strap therethrough that affixes the base plate to a planar surface in order to prevent movement of the base plate.

In order to facilitate movement of the footpad between the upright position and the stowed position, the base plate includes first and second base plate slots communicating respectively with first and second corner slots. The base plate further includes first and second locking slots configured to provide the upright position for the footpad and more preferably a plurality of first and second locking slots configured to provide the multiple upright positions for the footpad. The footpad includes first and second lower pins insertable respectively into the first and second base slots, thereby securing the footpad with the base plate. The footpad further includes first and second middle pins and first and second upper pins configured to fit respectively within the first and second locking slots and more preferably within one of the plurality of first and second locking slots.

The first and second lower pins in traversing respectively the first and second base slots moves the footpad from the stowed position to a partial deployment position. During a pivoting of the footpad, the first and second lower pins seat respectively within the first and second corner slots in order to move the footpad from the partial deployment position to the upright position. With the footpad in the upright position, the first and second middle pins insert respectively into the first and second locking slots thereby locking the footpad with the base plate in the upright position and more preferably into one of the plurality of the first and second locking slots thereby locking the footpad with the base plate in one of the multiple upright positions. Alternatively, the first and second middle pins in exiting respectively the first and second locking slots and more preferably one of the plurality of the first and second locking slots unlocks the footpad from the base plate. During a pivoting of the footpad, the first and second lower pins exit respectively the first and second corner slots in order to move the footpad from the upright position to the partial deployment position. From the partial deployment position, the first and second lower pins traverse respectively the first and second base plate slots thereby moving the footpad to the stowed position. The first and second upper pins in inserting respectively into the first and second locking slots and more preferably into one of the plurality of the first and second locking slots locks the footpad with the base plate in the stowed position.

The calfplate includes a heelcup configured to hold the foot in position atop the calfplate. The calfplate pivotally secures with the base plate such that the calfplate moves between a flat position and an elevated position. In order to maintain the calfplate in the elevated position, the base plate includes at least one swingout moveable between a stowed position and a support position, whereas the calfplate includes at least one elevator. When the swingout of the base plate resides in the support position, the elevator of the calfplate engages the swingout thereby locating the calfplate in the elevated position. Alternatively, when the swingout of the base plate resides in the stowed position, the elevator of the calfplate bypasses the swingout thereby locating the calfplate in the flat position. The calfplate includes a latch whereby, when the calfplate resides in the flat position, the latch engages the base plate thereby locking the calfplate with the base plate.

A method for creating a simulated foot position includes placing a foot atop a base, moving a footpad pivotally secured with the base to an upright position against the foot such that the footpad maintains the foot in a simulated foot position, and performing imaging of the foot. Placing a foot atop a base includes moving a calfplate of the base pivotally secured with a base plate of the base to an elevated position, and placing the foot atop the calfplate.

Problem(s) solved by this system include:
Weightbearing imaging is important to study the functional position of the foot for proper evaluation of the patient's anatomy.
During foot surgery, the patient is unable to stand for imaging that would show true weightbearing.
Physicians simulate weightbearing by pressing against the foot of the prone patient, with their hands or any available flat surface. This method is imprecise and not easily repeated. An imaging study may have differences in angles and loading.
There are currently no standards of care for consistent simulated weightbearing throughout orthopedics, or for non-ambulatory patients in any setting including the operating room, diagnostic imaging centers or physician offices. This system would allow for standards to be developed.
Healthcare professionals can use this system “hands-free”. Once set up, this system will provide the pressure necessary to simulate weightbearing hands-free (no active interaction) allowing the healthcare professionals to remove themselves from exposure.
This system creates reproducible positioning and orientation of a patient's foot. Images taken at different time points will show the same orientation and relative positioning of bones and joints which allows the physician to better visualize the effectiveness of their surgical intervention on the patient in a serial and consistent manner throughout an operation.

System improvements over existing technology include:
Current methods do not create reproducible simulated weightbearing positions. Current methods are manually applied, from different angles and with different amounts of pressure. Current methods are not reproducible if multiple x-rays are desired, at different stages during surgery and post-operatively. Other methods are for use after surgery, but not intraoperatively.
Current methods require ambulatory patients that can get simulated weightbearing x-rays by standing on a specially designed platform.
The current methods used to simulate weightbearing in most non-ambulatory settings require a healthcare professional to actively engage with the patient in order to hold their leg and foot in a proper position while the image is being taken thus exposing them to radiation.
The current methods used to simulate weightbearing frequently involve radiopaque and/or metal components, reducing the quality and accuracy of the image.

System purposes include:
To allow a physician or technician to effectively orient and maintain the foot of a patient in a simulated foot position such as a simulated weightbearing position and permit imaging in that position.
To allow this positioning to occur quickly, and reproducibly, such that the imaging studies show the foot in the same position each time.
To allow images to be taken throughout the diagnostic, procedural, and follow up setting in order to improve the accuracy of the information that physicians are currently using in foot and ankle and other orthopedics specialties.
To allow placement of a patient's foot in a simulated foot position such as a simulated weightbearing position, and maintained in that position, in a hands-free manner so that operating room or staff are not exposed to imaging energy.
It allows different angulations of the foot for simulated foot positions such as simulated weightbearing when desired by physician.
To elevate the foot being imaged above the patient's other leg so that there is no interference during lateral imaging.

System features include:
Fully radiolucent, except radiopaque marker strips allow for more accurate positioning and perspective on lateral radiograph images.
Hands-free (once set in place).
Reproducible.
Quick set and release.
Multi-use (autoclavable for operating room use, or non-sterile use).
Operates in different clinical settings: intraoperative, post-op, clinic.
Folds flat to a working surface.
Portable.
Allows entire foot visualization.
Allows elevation of the foot being imaged above the patient's other leg.
Eliminates the need to continuously manipulate the patient's leg and foot intraoperatively.
Patient comfort features such as rounded edges, cushions, etc.
Footpad able to adjust to various angles.
Can be used for A/P (anterior-posterior) or lateral imaging with no reassembly.
Footpad features include heel support, guiderails for foot, texture, and holes for cleaning.
Slots to receive belts for the table, leg, or foot, to stabilize the system.

Still other objects, features, and advantages of the present invention will become evident to those of ordinary skill in the art in light of the following. Also, it should be understood that the scope of this invention is intended to be broad, and any combination of any subset of the features, elements, or steps described herein is part of the intended scope of the invention.

### BRIEF DESCRIPTION OF THE DRAWINGS

FIG. 1 shows an isometric view of a simulated foot position system in position for patient use.

FIG. 2 shows a side view of a simulated foot position system in position for patient use, with a representative foot and leg.

FIG. 3 shows an isometric view of the base component of a simulated foot position system.

FIG. 4 shows an isometric view of a footpad for a simulated foot position system.

FIG. 5 shows the back end view of a footpad for a simulated foot position system.

FIG. 6 shows an isometric view of a calfplate for a simulated foot position system.

FIG. 7 shows an isometric close-up view of a calfplate detail.

FIG. 8 shows an isometric view of a simulated foot position system with the calfplate hidden.

FIG. 9 shows an isometric view of the footpad of a simulated foot position system in the upright position.

FIG. 10 shows an isometric view of a simulated foot position system during the process of being deployed.

FIG. 11 shows an isometric view of a simulated foot position system during a second position of being stowed.

FIG. 12 shows an isometric view of a simulated foot position system during a third position of being stowed.

FIG. 13 shows an isometric view of a simulated foot position system with footpad almost completely stowed.

FIG. 14 shows an isometric view of a simulated foot position system when it is stowed.

FIG. 15 shows a top view of a simulated foot position system when it is stowed.

FIG. 16 shows a side view of a simulated foot position system when it is stowed.

FIG. 17 shows an isometric view of a simulated foot position system when it is stowed, with the calf plate hidden.

FIG. 18 shows an isometric end view of a simulated foot position system when it is stowed.

FIG. 19 shows a side view of a simulated foot position system with footpad in the upright position.

FIG. 20 shows an end view of a simulated foot position system with footpad in the upright position and representative foot.

FIG. 21 shows an isometric rear view of a simulated foot position system with footpad in the upright position.

FIG. 22 shows an isometric view of a simulated foot position system with footpad in the upright position, showing radiopaque strips.

FIG. 23 shows a side view of a simulated foot position system with footpad in the upright position, showing radiopaque strips.

FIG. 24 shows an isometric view of a simulated foot position system with footpad in the stowed position and calfplate in the elevated position.

FIG. 25 shows a side view of a simulated foot position system with footpad in the stowed position and calfplate in the elevated position, with a representative foot and leg.

### DESCRIPTION OF COMPONENTS AND FEATURES

| Reference No. | Name | Main Purpose |
|---|---|---|
| 1 | simulated foot position | All the advantages described in this |
| | system, also shortened to sfp- | document. Simulated foot position such as |
| | system | simulated weightbearing can include various |
| | | amounts of load placed on the bottom of the |
| | | foot. |
| 10 | footpad upright position | The operating position for footpad 200, that |
| | | allows the physician to apply force to or load |
| | | the patient's foot for a weightbearing |
| | | radiograph. |
| 12 | footpad partial deployment | A series of interim positions of footpad 200 |
| | position | where lower pin 220 is not slipped into corner |
| | | slot 150 and moves along base slot 130. |
| | | These positions are transient when footpad |
| | | 200 is moving from upright position 10 to |
| | | stowed position 20. These positions can also |
| | | be used based on surgeon preference for the |
| | | imaging studies or surgery. |
| 20 | footpad stowed position | A stowed position of the footpad 200, that can |
| | | be used intraoperatively to keep footpad 200 |
| | | out of the way. The stowed position is also |
| | | for use when sfp-system 1 is being stored and |
| | | transported between uses. |
| 50 | calfplate elevated position | An elevated position of calfplate 300 that |
| | | allows patient's foot to be lifted for better |
| | | radiographs in a weightbearing position. |
| 55 | calfplate flat position | A flat position of calfplate 300 that allows |
| | | radiographs of patient's foot with less |
| | | separation from footpad 200 in Stowed |
| | | Position 20. |
| 75 | foot and leg | Representative patient foot and leg positioned |
| | | on Simulated Foot position system 1. |
| 100 | base | The main structure of the simulated foot |
| | | position system that includes base plate 105 |
| | | and calfplate 300 and attaches to footpad 200. |
| 105 | base plate | The lower portion of base 100 which provides |
| | | structural integrity and attaches to calfplate |
| | | 300 and footpad 200. |
| 110 | strap slots | Allow base 100 and base plate 105 to be |
| | | strapped or tethered to a hospital bed, |
| | | operating room table, or other flat surface. |
| | | Prevents movement of base 100 and base |
| | | plate 105. |
| 115 | base pin hole | Receives calfplate pin 410 and allows |
| | | calfplate 300 to rotate from flat to elevated |
| | | positions. |
| 120 | slot cutout | Allows footpad 200 to be partially rotated |
| | | while it is being pulled out of base plate 105. |
| | | Without slot cutout 120, footpad 200 would |
| | | have to be pulled straight out until it was away |
| | | from calfplate 300. |
| 130 | base slot | Horizontal slots in the base plate 105 that |
| | | allows lower pin 220 to slide along the base |
| | | plate 105 from nested position to upright |
| | | position. |
| 140 | base bottom | Prevents bedsheets from crumpling or |
| | | billowing into the way of footpad 200. |
| | | Ensures system can function. |
| 145 | base support | Insures strength and rigidity of base 100. |
| 150 | corner slot | A continuation of base slot 130 into a 90 |
| | | degree turn, such that lower pin 220 drops into |
| | | a locking position. |
| 160 | locking slots | The main purpose is to receive middle pin 225 |
| | | of footpad 200. There are multiple slots on |
| | | each side, to allow vertical upright positioning |
| | | of the footpad 200, or forward and rearward |
| | | angulation of footpad 200. |
| 170 | swingout notch | Allows swingout 400 to rotate into parallel |
| | | and perpendicular positions relative to base |
| | | 100. |
| 200 | footpad | Flat surface that applies pressure to the |
| | | bottom of a patient's foot when in the upright |
| | | position 10. Folds out of the way when in |
| | | stowed position 20. |
| 205 | foot strap slots | Allows patient's foot to be strapped or affixed |
| | | to footpad 200. |
| 210 | handle | Allows footpad 200 to be stowed and |
| | | deployed. Allows simulated foot position |
| | | system 1 to be carried and transported. |
| 220 | lower pin | Along with middle pin 225, allows footpad |
| | | 200 to be locked into an upright position. |
| | | Also allows footpad 200 to slide along base |
| | | slot 130. |
| 225 | middle pin | Allows footpad 200 to be locked into an |
| | | upright position. Fits into locking slots 160. |
| 230 | upper pin | Allows footpad 200 to be locked into locking |
| | | slot 160 for stowage and transport. |
| 240 | marker strips | Allows physician to see a simulated ground |
| | | on a radiograph when patient's foot is |
| | | positioned on footpad 200. Also allows |
| | | physician to orient the foot for a true lateral |
| | | radiograph. |
| 300 | calfplate | Supports the patient's leg and foot while the |
| | | system is in use. It can be in a flat position or |
| | | elevated position. The calfplate is part of base |
| | | 100. |
| 305 | heelcup | Allows patient's heel and lower calf to nestle |
| | | into the opening and be held in position for |
| | | radiographs. |
| 310 | latch | Allows calfplate 300 to be latched to base 100 |
| | | when footpad 200 is in stowed position, and |
| | | sfp-system 1 is to be carried or transported. |
| | | Latch 310 has a hook on the inside that snaps |
| | | over the edge of base 100. |
| 320 | elevator | When swingouts 400 are rotated |
| | | perpendicular to base 100, then right and left |
| | | elevator 320 engage swingouts 400 to hold |
| | | calfplate 300 in an elevated position. |
| 330 | calfplate pinholes | Allows calfplate pin 410 to attach calfplate |
| | | 300 to base 100, and allows calfplate 300 to |
| | | rotated relative to base 100. |
| 400 | swingout | Left and right swingouts 400 can allow |
| | | calfplate 300 to lie flat on base 100 when they |
| | | are in stowed or parallel to base 100. |
| | | swingouts 400 support calfplate 300 in |
| | | elevated position by engaging elevators 320 |
| | | when swingouts 400 are rotated 90 degrees. |
| 410 | calfplate pin | Attaches calfplate 300 to base 100. |

### DETAILED DESCRIPTION OF THE PREFERRED EMBODIMENT

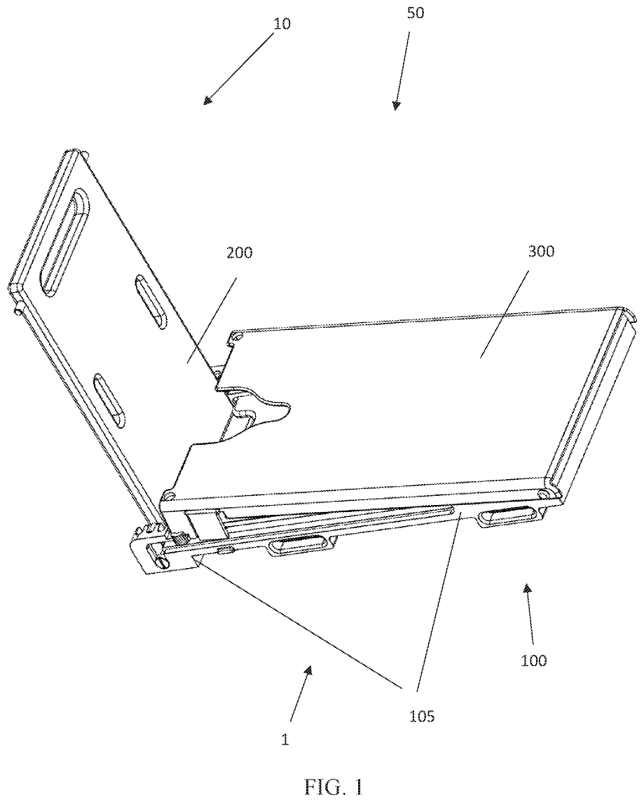

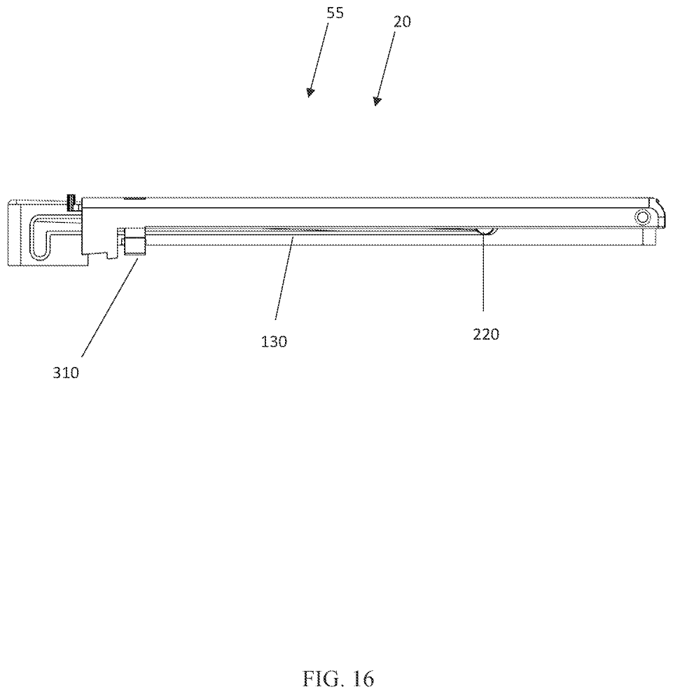

FIG. 1 displays a preferred embodiment of the simulated foot position system 1 (also called sfp-system 1). In the preferred embodiment, the simulated foot position provided by the sfp-system 1 simulates weightbearing, in which the physician or healthcare provider wishes to apply some amount of force to the bottom of the patient's foot. Major components include base 100 and footpad 200 which is shown in upright position 10. Base 100 includes base plate 105 and calfplate 300 which is shown in elevated position 50. Base plate 105 and calfplate 300 comprising the base 100 in the preferred embodiment include separate components secured in a pivotal relationship that allows movement of calfplate 300 between elevated position 50 and flat position 55 shown in FIG. 16. Nevertheless, one of ordinary skill in the art will recognize that the base 100 in a simpler configuration with less components may include base plate 105 and calfplate 300 manufactured as one piece with the calfplate 300 oriented in one of elevated position 50 and flat position 55. In the preferred embodiment, most or all of the components will be made of radiolucent materials, such as plastic, to minimize any artifacts during imaging.

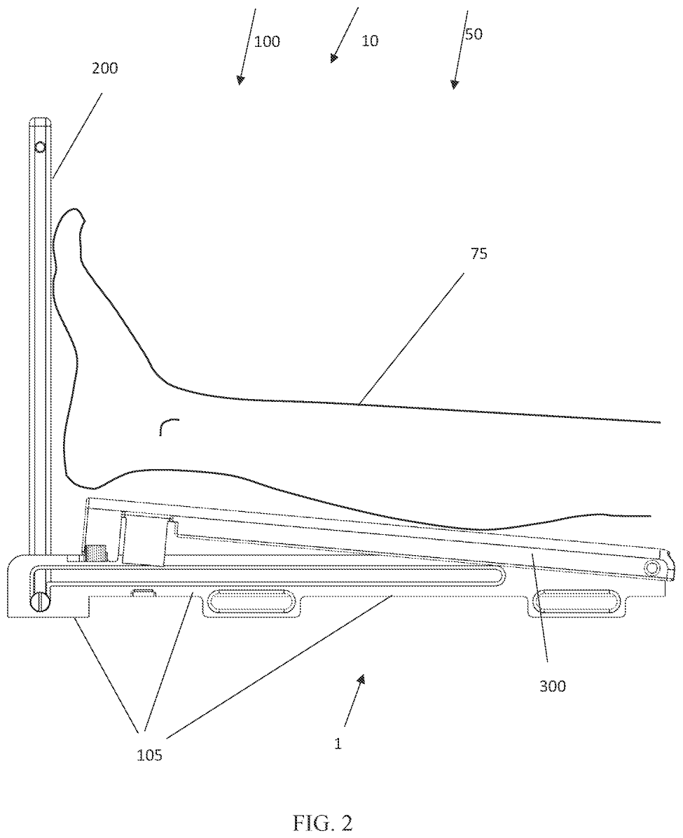

FIG. 2 displays a side view of sfp-system 1. In this figure, a typical patient's foot and leg 75 is shown resting on calfplate 300. The bottom of foot 75 contacts footpad 200. Footpad 200 pushes and holds foot 75 into the simulated foot position such as the simulated weightbearing position for imaging. In this position for the system, the patient's foot can be imaged in a repeatable and precise manner, without subjecting operating room staff to radiation.

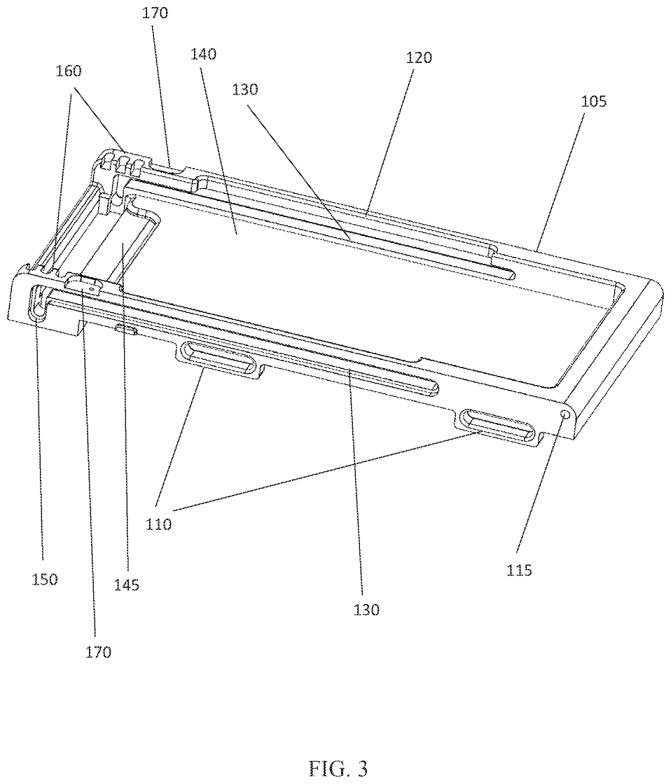

FIG. 3 shows an isometric view of base plate 105. Base plate 105 can be any shape and made of any material, but in this preferred embodiment is rectangular with approximate dimensions of 18″ long (along leg 10), 8″ wide, and 2″ tall. Base plate 105 is not solid; it includes a middle cavity that will allow nesting or stowage of footpad 200, similar to a drawer in a cabinet, as described later. Base bottom 140 is a flat piece on the bottom of base plate 105. Its purpose is to provide structural integrity to base plate 105, and also to prevent bedsheets and operating room materials from interfering with footpad 100 operation. One use of sfp-system 1 is on a hospital bed, and bedsheets billowing into the system would potentially interfere without base bottom 140. Base support 145 provides additional structural rigidity to base plate 105. There can be any number of base support 145 pieces, as needed. Strap slots 110 allow the base plate 105 to be strapped down to a hospital bed. Any type of strap can be fed through one or more strap slots 110 to firmly anchor base plate 105 to a hospital bed. It is apparent that by using strap slots 110, the base plate 105 can resist more force, and thus a greater force can be applied to the patient's foot so as to simulate a higher amount of weightbearing.

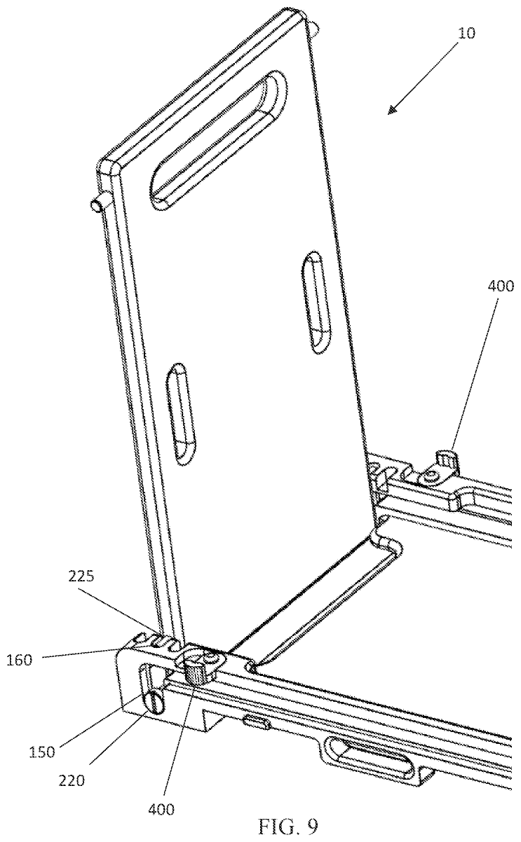

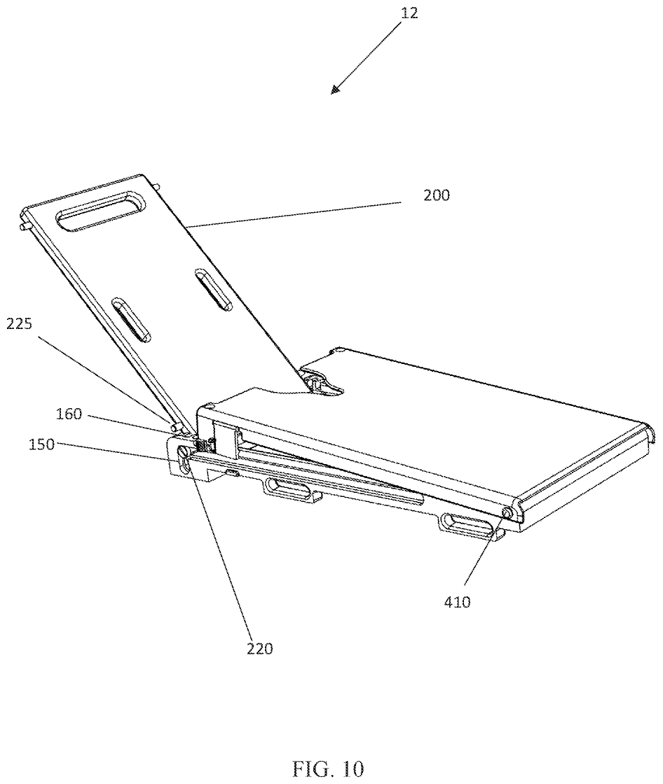

Base plate 105 includes two base slots 130, on each side of the base plate, that lie approximately horizontally. The purpose of base slots 130 is to allow footpad 200 to slide in and out, like a drawer, from a stowed position to an upright position or from an upright position to a stowed position. Slot cutout 120 is a notched area above base slot 130 that allows middle pin 225 to exit base slot 130. Middle pin 225 has dimensions smaller than lower pin 220, so that middle pin 225 can exit base slot 130 at slot cutout 120, whereas lower pin 220 cannot fit through slot cutout 120. In this way footpad 200 can rotate while it is being extended (for example a sequence shown in FIGS. 13 to 12 to 11 to 10), and will be discussed further later. Base slot 130 continues to the end of base plate 105 and then makes a right angle turn to corner slot 150. On the upper surface of base plate 105, near corner slot 150, are multiple locking slots 160. The preferred embodiment contains three sets of locking slots 160. One set of locking slots 160 is designed such that upright position 10 is anywhere from 45 degrees to 135 degrees, with a preferred embodiment of approximately 90 degrees relative to the calfplate 300 or footpad stowed position. Another set of locking slots 160 allows a forward-leaning position, and the third set for a rearward (reclined) leaning position from 0 to 75 degrees, with preferred embodiment at approximately 45 degrees. In each case, the angle is measured from the stowed horizontal position. In fact, it is apparent that there can be any number of sets of locking slots 160, one or more or even infinitely adjustable, as needed for the imaging procedure. Corner slot 150 and locking slots 160 work in conjunction with lower pin 220 and middle pin 225, to lock the footpad in upright position 10. As shown in FIG. 9, a couple moment is created by the separation distance from middle pins 225 and lower pins 220, with middle pins nestled in one set of locking slots 160 and lower pins 220 nestled in corner slot 150. This couple moment allows footpad 200 to lock into a stable upright position 10. When footpad 200 is locked into the upright position 10, then sfp-system 1 is hands-free, and pressure is applied to the patient's foot. A physician or technician can leave the path of the radiation knowing the patient's foot is in the proper position, and in a reproducible position. Base 100 can also have holes or openings in the bottom surface that would allow the system to be more easily cleaned after surgery.

By having multiple sets of locking slots 160, the surgeon has an option to position the patient's operative foot at different angles relative to base 100. Certain surgical procedures might require imaging in a simulated foot position such as a simulated weightbearing position in a non-orthogonal position such as dorsiflexion (foot angled towards head) or plantar flexion (foot angled away from head), for example in an athlete or dancer or similar occupation. A physician could even use multiple positions during an imaging study, each of which is held steady and repeatable by sfp-system 1.

Other features of base plate 105 are base pin hole 115 and swingout notch 170. Base pin hole 115 is a hole in base plate 105 that receives calfplate pin 410 and allows calfplate 300 to rotate from flat to elevated positions. Swingout notch 170 is a recessed area in base plate 105 that allows swingouts 400 to rotate into a nested position for when calfplate 300 is in calfplate flat position 55, or swingouts 400 can support calfplate 300 in elevated position 55 by engaging elevators 320 when swingouts 400 are rotated 90 degrees.

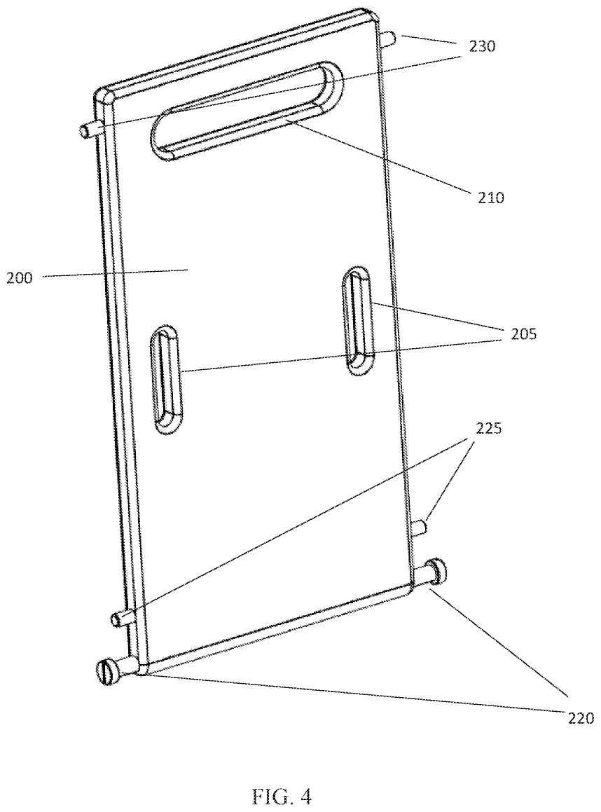

FIG. 4 shows an isometric frontal view of footpad 200. Footpad 200 can be any shape and made of any material, but in this preferred embodiment it is rectangular with approximate dimensions of 15″ tall, 6″ wide, and ¾″ thick, and is made of radiolucent plastic. Smaller or larger versions of the system can be created for different size patients. Foot strap slots 205 allow hospital staff to strap or attach a patient's foot to footpad 200, as desired. Handle 210 serves two purposes: (1) it can be used by a physician to extend and stow footpad 200 into base 100, and (2) when simulated foot position system 1 is in the fully stowed and locked position then handle 210 can be used to conveniently carry the system. Lower pins 220 slide in base slot 130 and then descend into corner slot 150. Middle pins 225, both shorter and smaller in diameter than lower pins 220, can exit base slot 130 at the location of slot cutout 120. Upper pins 230 are used to lock footpad 200 in a stowed position for storage and transport. Footpad 200 can also have a textured finish, to create more friction for the patient's foot, or a smooth finish. Guiderails could be added footpad 200 to help stabilize the patient's foot.

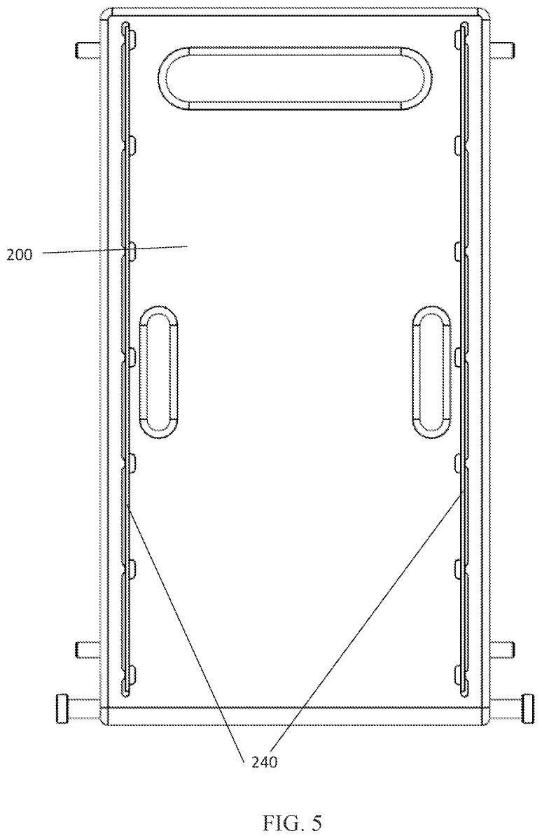

FIG. 5 shows an isometric rear view of footpad 200. In this view, marker strips 240 are visible. Marker strips 240 can be made of a radiopaque material, such as metal, and affixed to the perimeter of footpad 200, so that a physician has a reference for orientation and dimensions when imaging. Marker strips 240 can be a continuous perimeter of radiopaque material, or can be smaller pieces that are only in certain locations on the footpad 200. Marker strips 240 can also be overmolded, glued, or snapped into place on footpad 200. In the preferred embodiment, marker strips 200 are embedded in cavities in the rear of footpad 200, so that they lie close to the front surface of footpad 200. In that location, the patient cannot contact marker strips 240, however marker strips 240 will appear in any imaging. By having two marker strips 240, a physician can use the marker strips to help align the patient for a true lateral radiograph. In a true lateral radiograph, the two marker strips would appear as only one, since the distal strip would be hidden by the proximal strip.

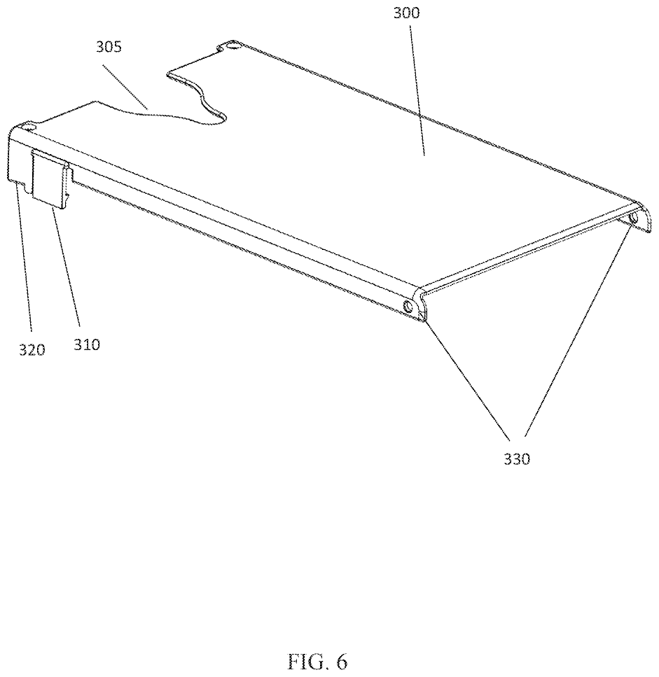

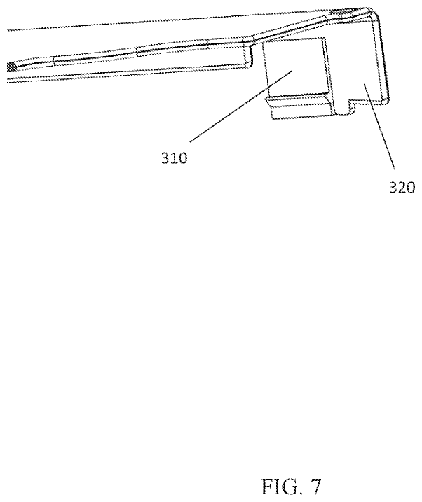

FIG. 6 shows calfplate 300 in an isometric top view. Calfplate 300 is the upper part of base 100 and is designed for a patient's foot and lower calf to rest. Calfplate 300 has calfplate pinholes 330 at one end that allow for attachment to base plate 105, and also allow for rotation of calfplate 300 relative to base plate 105. Heelcup 305 is an optional feature that provides an anatomically conforming location to position the patient's heel and lower extremity. By nestling the heel at this location, the user is assured that footpad 200 will contact the plantar surface of the foot when footpad 200 is in the upright position 10. Latch 310 on footpad 300 is flexible and has a hook on the inside. When calfplate 300 is lowered to a flat position relative to base plate 105, then latch 310 can hook onto an edge of base plate 105 and thus lock calfplate 300 in place, for storage and transport. Elevators 320 are used to support calfplate 300 in elevated position 50. Elevators 320 can rest on swingouts 400 when they are rotated perpendicular to base plate 105, and thus the patient's foot and leg are elevated away from base plate 105. This is a useful addition to (1) permit easier access to the patient's foot, and (2) allow the foot being operated on to rise out of the view from the non-operative foot during a lateral image. Without the elevated position, a physician might have to move or bend the non-operative foot at the knee to remove it from a lateral image. Calfplate 300 has two positions, elevated position 50 and flat position 55. Either position is suitable for use during a surgical procedure, depending on physician needs, and other incremental elevated positions are also possible in alternate embodiments. FIG. 7 is a close-up outward-looking view of latch 310 and elevator 320.

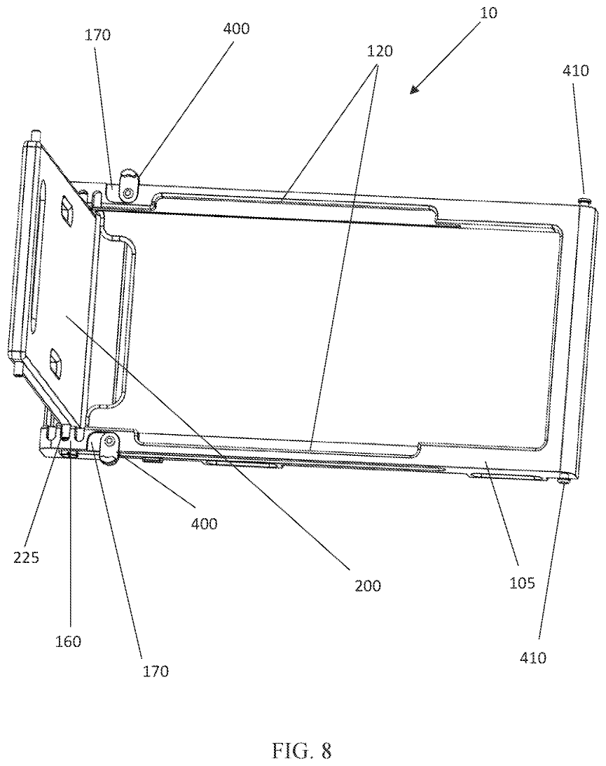

FIG. 8 shows swingout 400, along with base plate 105 and footpad 200. Calfplate 300 is not shown (hidden) in FIG. 8 to simplify the figure. Swingout 400 is a set of two pieces that attach to base plate 105. In the parallel position, they lie parallel to base slot 130, and calfplate 300 passes alongside swingouts 400 without contact when it is lowered to a flat position. In the perpendicular position as shown in FIGS. 8 and 9, when swingouts 400 are swung out to 90 degrees, they provide a resting surface for elevators 320, holding calfplate in elevated position 50.

FIG. 9 shows a close up isometric view of base plate 105 and footpad 200, with calfplate 300 hidden for simplicity. The functioning of swingouts 400 is more apparent in this figure. In addition, the interaction of middle pin 225 with locking slots 160, and lower pin 220 with corner slot 150 is more apparent.

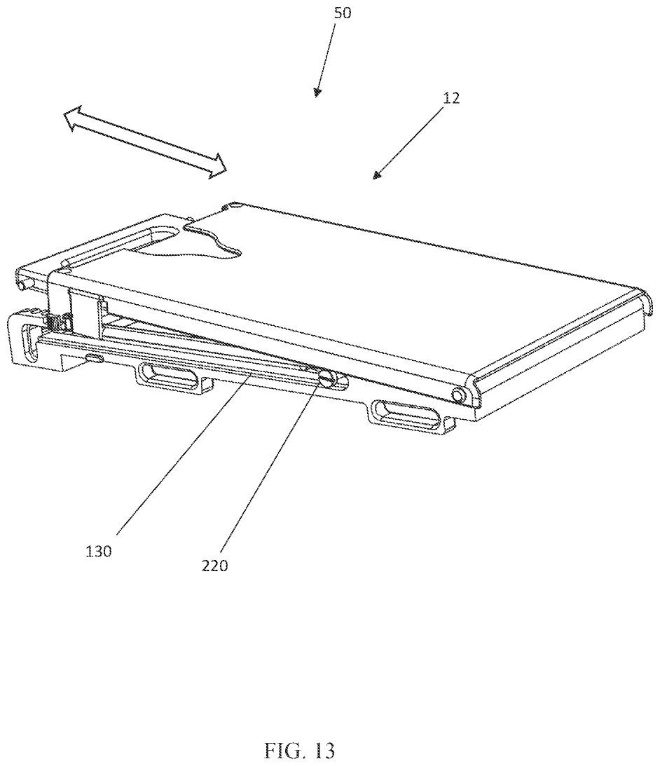

FIGS. 9 through 13 show the simulated foot position system as footpad 200 is being transitioned from upright position 10 to stowed position 20. Looking at the figures in reverse, FIGS. 13 through 9, would also represent the motion from stowed position 20 to upright position 10. This sequence of motions can occur back and forth during a surgical procedure, as many times as desired by the physician. The physician can conveniently leave the patient's operative foot and leg in position on the calfplate throughout the footpad motions, and can even perform surgery on the foot while in left place on sfp-system 1.

FIG. 10 shows simulated foot position system 1 as footpad 200 is being transitioned between upright position 10 and stowed position 20. In FIG. 10, footpad 200 has been lifted or raised using handle 210 such that lower pins 220 are no longer at the bottom of corner slots 150. Similarly, middle pins 225 are no longer in locking slots 160. With no resisting couple moment between these sets of pins, footpad 200 can be rotated rearward into partial deployment position 12. Partial deployment position 12 is a transient set of positions that lie between upright position 10 and stowed position 20. Calfplate pin 410 can be seen passing through calfplate pinholes 330 and thence into base pin holes 115, thus attaching calfplate 300 to base plate 105 such that calfplate 300 can rotate relative to base plate 105.

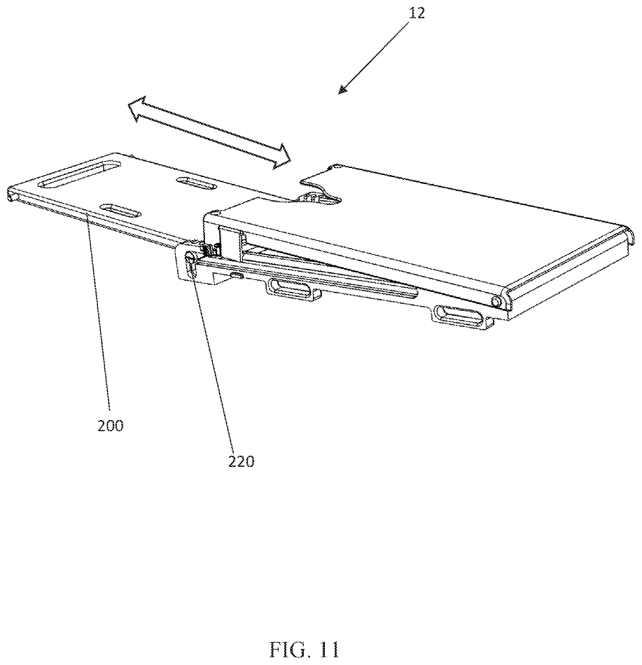

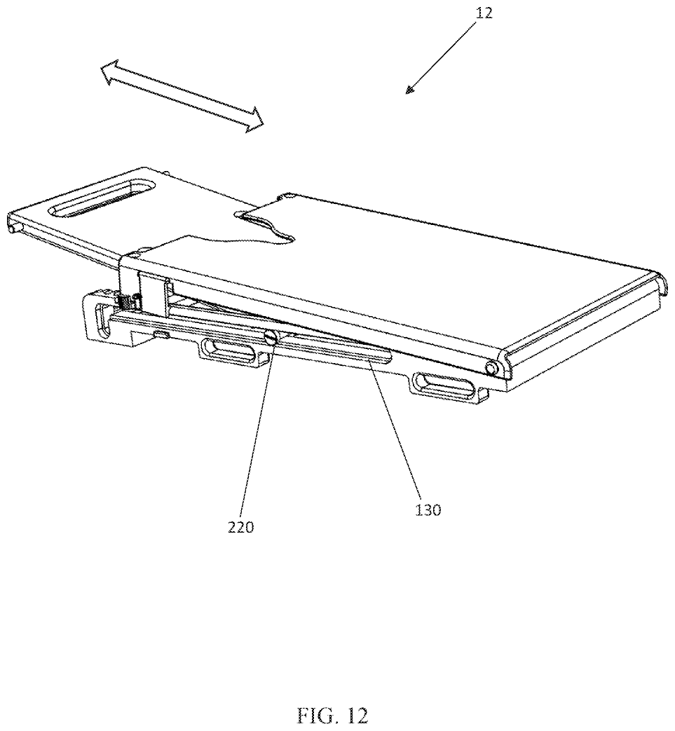

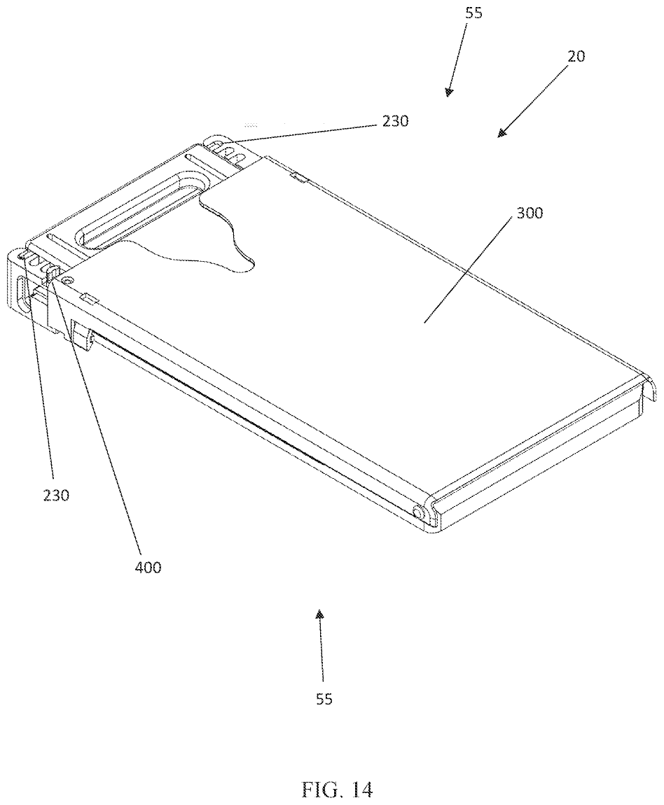

FIG. 11 shows sfp-system 1 as it is further transitioned during operation. Footpad 200 has now been rotated to nearly flat. FIG. 12 then shows the user moving handle 210 inwards, such that lower pin 220 slides in base channel 130, under calfplate 300. Since the patient's foot and leg is on top of calfplate 300, and since base bottom 140 is on the bottom of base plate 105, there is an unimpeded ability for footpad 200 to slide into a hidden stowed position under calfplate 300. FIG. 13 shows footpad 200 as lower pin 220 has now reached the end of travel in base slot 130. In FIGS. 11-13, calfplate 300 is resting on swingouts 400 in elevated position 50. Alternatively, the physician system could leave the calfplate 300 in Flat Position 55 if preferable for a given patient. In a final step, FIG. 14 shows an isometric view of upper pins 230 placed into locking slots 160, so that footpad 200 can no longer slide in or out. Likewise in FIG. 14, the swingouts 400 have been rotated into base plate 105, such that calfplate 300 now falls into flat position 55.

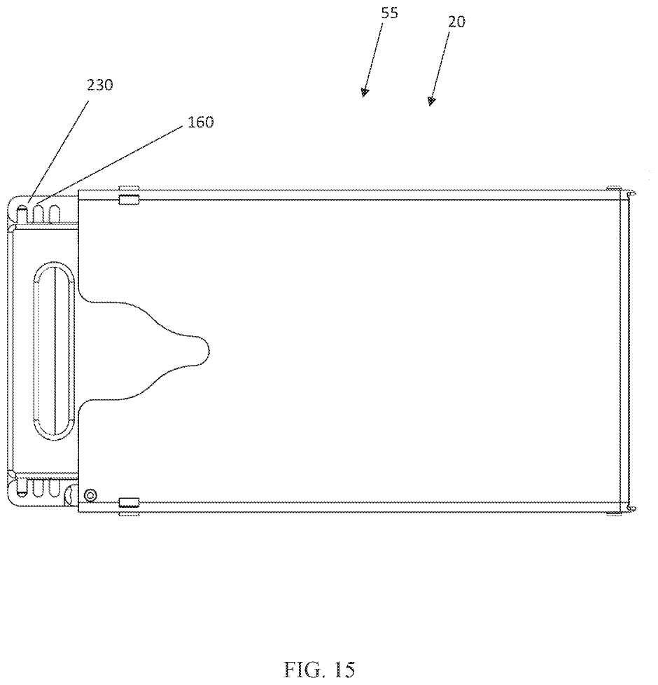

FIGS. 15 and 16 show top and side views of footpad 200 in stowed position 20, and calfplate 300 in flat position 55. Latch 310 now snaps over the edge of base plate 105 keeping calfplate locked in place. Upper pins 230 are snug in locking slots 160, keeping the footpad 200 in place. The sfp-system 1 is now able to be stored or transported with pieces held in a stable configuration. FIG. 16 shows the utility of the sfp-system 1. In this stowed position, a physician standing at the end of an operating room bed can perform surgery on the foot while it lies on the sfp-system 1. There is no need to remove and reposition system 1 under the patient during surgery, thus maximizing efficiency. There is also no need to move the non-operative foot up or down or bend it at the knee, to keep it out of lateral images—this is accomplished by the elevated position of the calfplate. Whenever an image is needed, footpad 200 is deployed to the upright position 10.

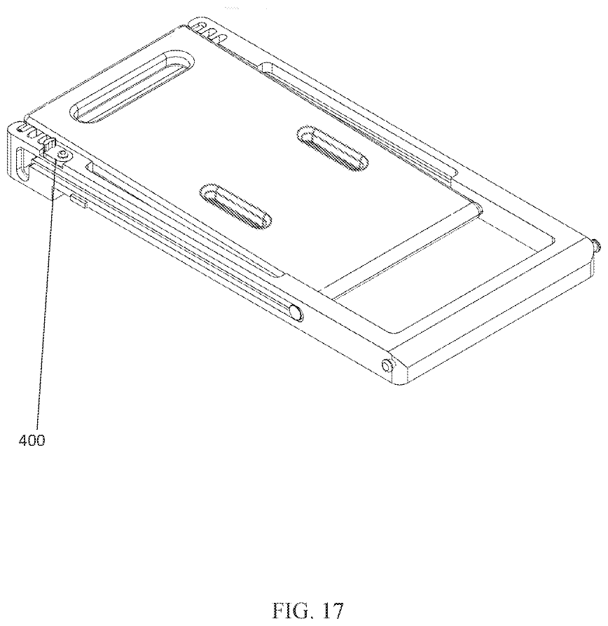

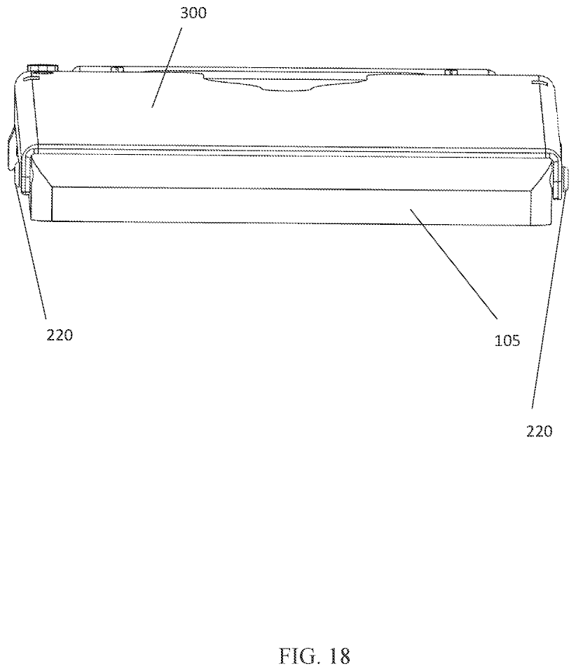

FIG. 17 is an isometric with footpad 200 in stowed Position 20, and calfplate 300 hidden for clarity. It is now easily visible how footpad 200 fits in the cavity of base 100, situated between base plate 105 and calfplate 300. FIG. 18 is an end view of sfp-system 1 in the storage and stowed position.

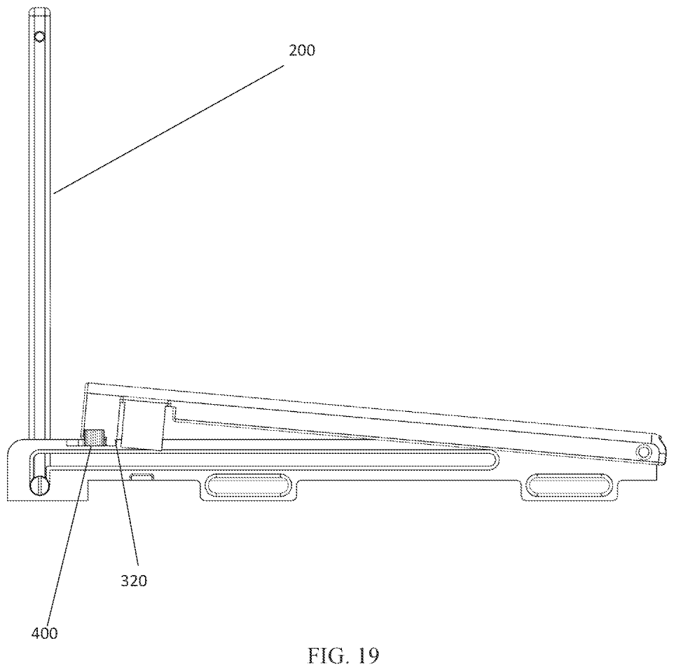

FIG. 19 is a side view of sfp-system 1 drawing attention to elevator 320 contacting swingout 400, while swingout 400 is rotated away from base plate 105.

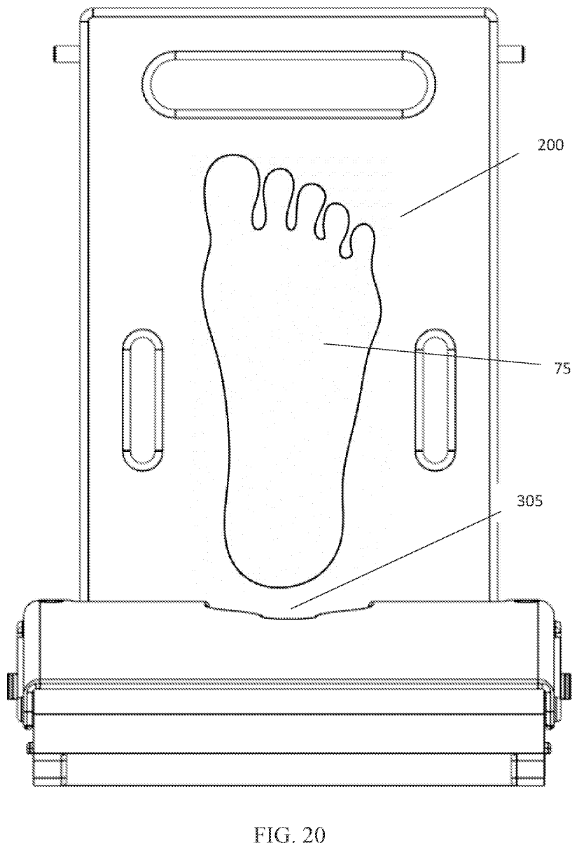

FIG. 20 is an end view of sfp-system 1 in upright position 10 and elevated position 50, with representative foot and leg 75 contacting footpad 200. In use, the heel of foot 75 would nestle into heelcup 305. Heelcup 305 could have foam, rubber, towels, or other soft materials to gently contact foot 75. If desired, foot strap slots 205 can be used to hold foot 75 in an upright or angled position.

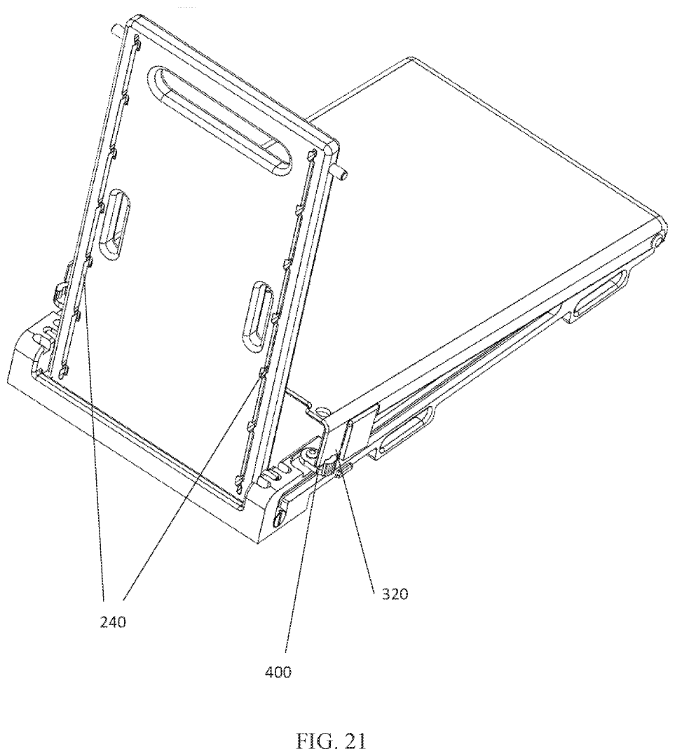

FIG. 21 is an isometric rear facing view of sfp-system 1 to show various features, middle pins 225 are in locking slots 160, and lower pins 220 are in corner slot 150 in this figure, thus locking footpad 200 in upright position 10.

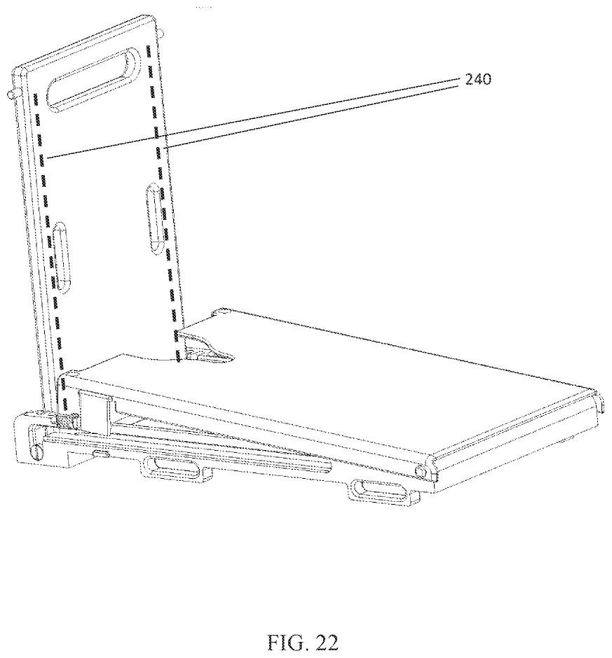

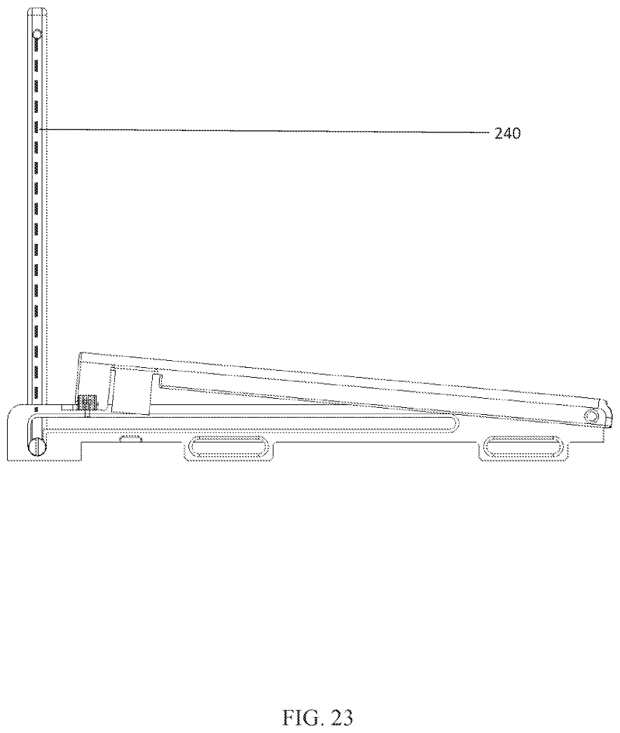

FIGS. 22-23 are shaded to better show how marker strips 240 operate. Marker strips 240 are the only radiopaque items in sfp-system 1. They are embedded either in slots of footpad 200, or overmolded, glued or otherwise adhered. There can be two marker strips as shown in the preferred embodiment, or any number of strips if certain markings or a grid pattern is desired. In FIG. 23, it is apparent that marker strips 240 will be visible on a lateral radiograph, and represent ground for the patient's foot. A physician can use marker strips 240 to insure a true lateral radiograph is taken, thus creating more precision during imaging.

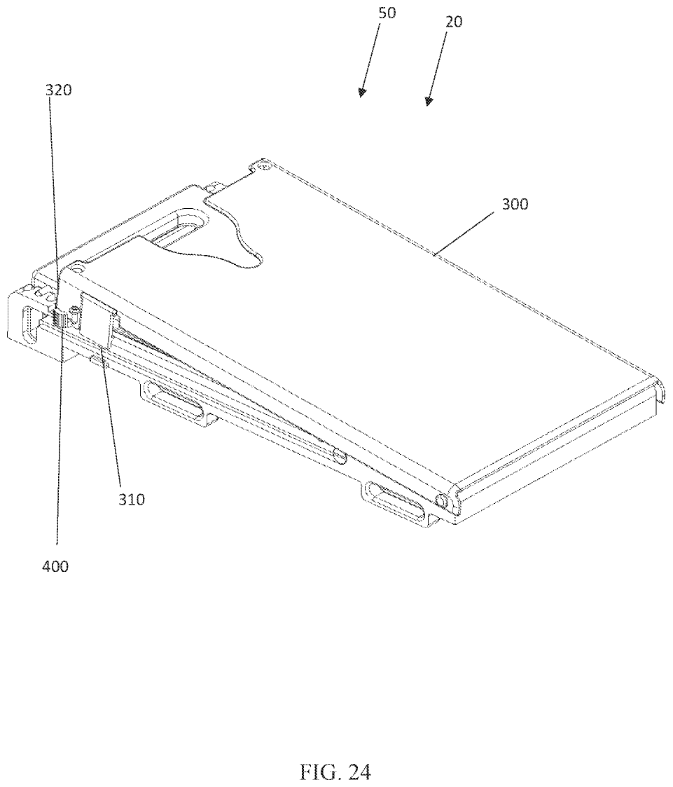

FIG. 24 is an isometric view of sfp-system 1 with footpad in stowed position 20 and calfplate in elevated position 50. In this configuration, the system can be positioned under the patient's operative foot and leg, with heel in or near heelcup 305.

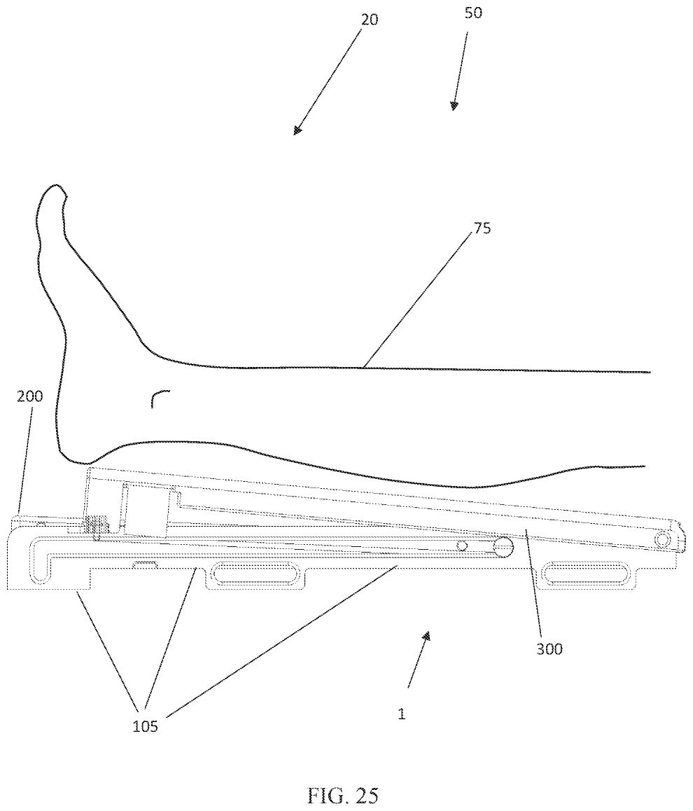

FIG. 25 is a side view of sfp-system 1 with footpad in stowed position 20 and calfplate in elevated position 50. In this configuration, the system can be positioned under the patient's operative foot and leg 75, with heel in or near heelcup 305. Foot and leg 75 also rests on calfplate 300.

The simulated foot position system includes the following features.

Features of the system include:
Fully radiolucent except marker strips on footpad to simulate ground during lateral imaging.
Hands-free (once locked in place in upright position), which prevents unwanted exposure to operating room staff.
Reproducible and repeatable imaging by having a flat surface (footpad) pressing on the foot at a known angle (upright position).
Quick stowing, deployment, and locking of the footpad as many times as needed during a surgical procedure or imaging study.
Multi-use (autoclavable for operating room use, or non-sterile use).
Operates in different clinical settings: intraoperative, post-op, clinic.
Folds flat to a working surface, such as FIG. 16 or 24.
Portable, with a handle and latches that hold the parts together during transport.
Allows entire foot visualization from lateral or anterior-posterior (A-P).
Allows foot being imaged to be elevated above non-operative patient foot, to avoid artifacts during lateral images.
Allows the operative foot to be elevated to make it easier for physician to access during surgery.
Patient comfort features such as rounded edges, cushions, etc.
Footpad able to adjust to various angles, at the convenience of the surgeon.
Can be used for A/P or lateral imaging with no reassembly.
Footpad features include heel support, guiderails for foot, texture, and holes for cleaning.
Heelcup receives the heel of the operative foot and holds it steady during surgery or imaging.
Slots to receive belts or straps that allow stabilizing the simulated foot position system and patient's leg and foot during the procedures.

A method of use for using the simulated foot position system in surgery is described here. A patient is prepped for surgery according to physician's instructions. A sterile field is established. The simulated foot position system can be sterilized, if desired. The sterile sfp-system 1 in collapsed and latched position is placed at end of bed; FIGS. 14-16 show the system in stowed and latched position. Base plate 105 rests on the bed to provide structural integrity and connections to calfplate 300 and footpad 200. The calfplate 300 is moved to the elevated position 50 by rotating swingouts 400 into outward positions, and then resting elevators 320 on swingouts 400 as shown in FIG. 24. Optionally, calfplate 300 can be left in flat position 55 if preferred. Then a patient's foot and leg is placed on top of the system, with footpad 200 in stowed position 20 and calfplate 300 in elevated position 50, as shown in FIG. 25. The patient's heel is placed in heelcup 305. When desired, the footpad 200 is deployed to upright position 10, as shown in the sequence of FIGS. 9-13. Images are obtained at the preference of the physician. The footpad 200 is then collapsed and re-stowed into the nested position 20. This cycle of deploying and nesting the system can be repeated any number of times during a surgical procedure. The physician can perform surgery on the foot in any of the positions, whichever is most convenient for him or her. In the preferred embodiment, the physician leaves the patient's foot in the elevated position 50 with footpad in stowed position 20 while performing surgery, and uses upright position 20 for imaging.

A method of use for using the simulated foot position system with a non-sedated but prone patient is described here. In this case there is no sterile field. A patient is situated on a table in a lying position. The non-sterile simulated foot position system 1 in stowed position is placed at end of bed. Patient's leg is placed on top of stowed system on calfplate 300. Patient's leg can be belted to the base and/or footpad of simulated foot position system, to help with stability. When desired, the footpad is deployed. Imaging studies are performed. The footpad is then stowed into the nested position. This cycle of deploying and nesting the system can be repeated any number of times during a clinical visit.

Although the present invention has been described in terms of the foregoing preferred embodiment, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

What is claimed is:

1. A system for creating a simulated foot position, comprising:
a base being configured to support a foot thereon; and
a footpad securable with the base in an upright position, the footpad being configured to receive the foot thereagainst, whereby the footpad maintains the foot in a simulated foot position.

2. The system for creating a simulated foot position of claim 1, the footpad being pivotally securable with the base such that the footpad moves between the upright position and a stowed position whereby the footpad inserts within the base.

3. The system for creating a simulated foot position of claim 2, wherein:
the base, including:
at least a first base slot communicating with a first corner slot, and
at least a first locking slot configured to provide the upright position for the footpad; and
the footpad including:
at least a first lower pin insertable into the first base slot, thereby securing the footpad with the base, and
at least a first middle pin configured to fit within the first locking slot.

4. The system for creating a simulated foot position of claim 3, wherein:
the first lower pin traversing the first base slot moves the footpad from the stowed position to a partial deployment position;
the footpad pivoting while the first lower pin seats within the first corner slot moves the footpad from the partial deployment position to the upright position; and
the first middle pin inserting into the first locking slot locks the footpad with the base in the upright position.

5. The system for creating a simulated foot position of claim 4, the footpad including at least a first upper pin configured to fit within the first locking slot.

6. The system for creating a simulated foot position of claim 5, wherein:
the first middle pin exiting the first locking slot unlocks the footpad from the base;
the footpad pivoting while the first lower pin exits the first corner slot moves the footpad from the upright position to the partial deployment position;
the first lower pin traversing the first base slot moves the footpad from the partial deployment position to the stowed position; and
the first upper pin inserting into the first locking slot locks the footpad with the base in the stowed position.

7. The system for creating a simulated foot position of claim 3, wherein:
the base, including:
a second base slot communicating with a second corner slot opposite from the first base slot, and
a second locking slot opposite from the first locking slot, the second locking slot being configured to provide the upright position for the footpad; and
the footpad including:
a second lower pin opposite from the first lower pin, the second lower pin being insertable into the second base slot, thereby securing the footpad with the base; and
a second middle pin opposite from the first middle pin, the second middle pin being configured to fit within the second locking slot.

8. The system for creating a simulated foot position of claim 7, wherein:
the first lower pin and the second lower pin traversing respectively the first base slot and the second base slot moves the footpad from the stowed position to a partial deployment position;
the footpad pivoting while the first lower pin and the second lower pin seat respectively within the first and second corner slots moves the footpad from the partial deployment position to the upright position; and
the first middle pin and the second middle pin inserting respectively into the first and second locking slots locks the footpad with the base in the upright position.

9. The system for creating a simulated foot position of claim 8, the footpad including a second upper pin opposite from the first upper pin, the second upper pin configured to fit within the second locking slot.

10. The system for creating a simulated foot position of claim 9, wherein:
the first middle pin and the second middle pin exiting respectively the first and second locking slots unlocks the footpad from the base;
the footpad pivoting while the first lower pin and the second lower pin exit respectively the first and second corner slots moves the footpad from the upright position to the partial deployment position;
the first lower pin and the second lower pin traversing respectively the first base slot and the second base slot moves the footpad from the partial deployment position to the stowed position; and
the first upper pin and the second upper pin inserting respectively into the first and second locking slots locks the footpad with the base in the stowed position.

11. The system for creating a simulated foot position of claim 1, the footpad being pivotally securable with the base such that the upright position of the footpad includes multiple upright positions having different angles that provide the footpad with multiple simulated foot positions.

12. The system for creating a simulated foot position of claim 11, wherein:
the base, including a plurality of first locking slots configured to provide the multiple upright positions for the footpad; and
the footpad including:
at least a first lower pin pivotally securing the footpad with the base, and
at least a first middle pin configured to fit within one of the plurality of first locking slots.

13. The system for creating a simulated foot position of claim 12, wherein:
the footpad pivoting about the base moves the footpad among the multiple upright positions; and
the first middle pin inserting into one of the plurality of first locking slots locks the footpad with the base in one of the multiple upright positions.

14. The system for creating a simulated foot position of claim 12, wherein:
the base, including a plurality of second locking slots opposite from the plurality of first locking slots, the plurality of second locking slots being configured to provide the multiple upright positions for the footpad; and
the footpad including:
a second lower pin opposite from the first lower pin, the second lower pin pivotally securing the footpad with the base, and
a second middle pin opposite from the first middle pin, the second middle pin being configured to fit within one of the plurality of second locking slots.

15. The system for creating a simulated foot position of claim 14, wherein:
the footpad pivoting about the base moves the footpad among the multiple upright positions; and
the first middle pin and the second middle pin inserting respectively into one of the plurality of first locking slots and second locking slots locks the footpad with the base in one of the multiple upright positions.

16. The system for creating a simulated foot position of claim 1, wherein the simulated foot position comprises a simulated weightbearing foot position.

17. The system for creating a simulated foot position of claim 1, the footpad including at least one radiopaque trim piece.

18. The system for creating a simulated foot position of claim 1, the footpad including a first strap slot and a second strap slot configured to receive a strap therethrough that affixes the foot to the footpad.

19. The system for creating a simulated foot position of claim 1, the base including a heelcup configured to hold the foot in position atop the base.

20. The system for creating a simulated foot position of claim 1, the base including a first strap slot and a second strap slot configured to receive a strap therethrough that affixes the base to a planar surface that prevents movement of the base.

21. The system for creating a simulated foot position of claim 1, the base, comprising:
a base plate; and
a calfplate securable with the base plate, the calfplate being configured to support the foot thereon.

22. The system for creating a simulated foot position of claim 21, the calfplate including a heelcup configured to hold the foot in position atop the calfplate.

23. The system for creating a simulated foot position of claim 21, the calfplate being pivotally securable with the base plate, whereby the calfplate moves between a flat position and an elevated position.

24. The system for creating a simulated foot position of claim 23, wherein:
the base plate including at least one swingout moveable between a stowed position and a support position; and
the calfplate including at least one elevator, whereby, when the swingout of the base plate resides in the support position, the elevator of the calfplate engages the swingout thereby locating the calfplate in the elevated position, and, when the swingout of the base plate resides in the stowed position, the elevator of the calfplate bypasses the swingout thereby locating the calfplate in the flat position.

25. The system for creating a simulated foot position of claim 23, the calfplate including a latch whereby, when the calfplate resides in the flat position, the latch engages the base plate thereby locking the calfplate with the base plate.

26. A system for creating a simulated foot position, comprising:
a base plate;
a calfplate securable with the base plate, the calfplate being configured to support a foot thereon; and
a footpad securable with the base plate in an upright position, the footpad being configured to receive the foot thereagainst, whereby the footpad maintains the foot in a simulated foot position.

27. The system for creating a simulated foot position of claim 26, the footpad being pivotally securable with the base plate such that the footpad moves between the upright position and a stowed position whereby the footpad inserts between the base plate and the calfplate.

28. The system for creating a simulated foot position of claim 27, wherein:
the base plate, including:
at least a first base slot communicating with a first corner slot, and
at least a first locking slot configured to provide the upright position for the footpad; and
the footpad including:
at least a first lower pin insertable into the first base slot, thereby securing the footpad with the base plate, and
at least a first middle pin configured to fit within the first locking slot.

29. The system for creating a simulated foot position of claim 28, wherein:
the first lower pin traversing the first base slot moves the footpad from the stowed position to a partial deployment position;
the footpad pivoting while the first lower pin seats within the first corner slot moves the footpad from the partial deployment position to the upright position; and
the first middle pin inserting into the first locking slot locks the footpad with the base plate in the upright position.

30. The system for creating a simulated foot position of claim 29, the footpad including at least a first upper pin configured to fit within the first locking slot.

31. The system for creating a simulated foot position of claim 30, wherein:
the first middle pin exiting the first locking slot unlocks the footpad from the base plate;
the footpad pivoting while the first lower pin exits the first corner slot moves the footpad from the upright position to the partial deployment position;
the first lower pin traversing the first base slot moves the footpad from the partial deployment position to the stowed position; and
the first upper pin inserting into the first locking slot locks the footpad with the base plate in the stowed position.

32. The system for creating a simulated foot position of claim 28, wherein:
the base plate, including:
a second base slot communicating with a second corner slot opposite from the first base slot, and
a second locking slot opposite from the first locking slot, the second locking slot being configured to provide the upright position for the footpad; and
the footpad including:
a second lower pin opposite from the first lower pin, the second lower pin being insertable into the second base slot, thereby securing the footpad with the base plate; and
a second middle pin opposite from the first middle pin, the second middle pin being configured to fit within the second locking slot.

33. The system for creating a simulated foot position of claim 32, wherein:
the first lower pin and the second lower pin traversing respectively the first base slot and the second base slot moves the footpad from the stowed position to a partial deployment position;
the footpad pivoting while the first lower pin and the second lower pin seat respectively within the first and second corner slots moves the footpad from the partial deployment position to the upright position; and
the first middle pin and the second middle pin inserting respectively into the first and second locking slots locks the footpad with the base plate in the upright position.

34. The system for creating a simulated foot position of claim 33 the footpad including a second upper pin opposite from the first upper pin, the second upper pin configured to fit within the second locking slot.

35. The system for creating a simulated foot position of claim 34, wherein:
the first middle pin and the second middle pin exiting respectively the first and second locking slots unlocks the footpad from the base plate;
the footpad pivoting while the first lower pin and the second lower pin exit respectively the first and second corner slots moves the footpad from the upright position to the partial deployment position;
the first lower pin and the second lower pin traversing respectively the first base slot and the second base slot moves the footpad from the partial deployment position to the stowed position; and
the first upper pin and the second upper pin inserting respectively into the first and second locking slots locks the footpad with the base plate in the stowed position.

36. The system for creating a simulated foot position of claim 26, the footpad being pivotally securable with the base plate such that the upright position of the footpad includes multiple upright positions having different angles that provide the footpad with multiple simulated foot positions.

37. The system for creating a simulated foot position of claim 36, wherein:
the base plate, including a plurality of first locking slots configured to provide the multiple upright positions for the footpad; and
the footpad including:
at least a first lower pin pivotally securing the footpad with the base plate, and
at least a first middle pin configured to fit within one of the plurality of first locking slots.

38. The system for creating a simulated foot position of claim 37, wherein:
the footpad pivoting about the base plate moves the footpad among the multiple upright positions; and
the first middle pin inserting into one of the plurality of first locking slots locks the footpad with the base plate in one of the multiple upright positions.

39. The system for creating a simulated foot position of claim 37, wherein:
the base plate, including a plurality of second locking slots opposite from the plurality of first locking slots, the plurality of second locking slots being configured to provide the multiple upright positions for the footpad; and
the footpad including:
a second lower pin opposite from the first lower pin, the second lower pin pivotally securing the footpad with the base plate, and
a second middle pin opposite from the first middle pin, the second middle pin being configured to fit within one of the plurality of second locking slots.

40. The system for creating a simulated foot position of claim 39, wherein:
the footpad pivoting about the base plate moves the footpad among the multiple upright positions; and
the first middle pin and the second middle pin inserting respectively into one of the plurality of first locking slots and second locking slots locks the footpad with the base plate in one of the multiple upright positions.

41. The system for creating a simulated foot position of claim 26, wherein the simulated foot position comprises a simulated weightbearing foot position.

42. The system for creating a simulated foot position of claim 26, the footpad including at least one radiopaque trim piece.

43. The system for creating a simulated foot position of claim 26, the footpad including a first strap slot and a second strap slot configured to receive a strap therethrough that affixes the foot to the footpad.

44. The system for creating a simulated foot position of claim 26, the base plate including a heelcup configured to hold the foot in position atop the base plate.

45. The system for creating a simulated foot position of claim 26, the base plate including a first strap slot and a second strap slot configured to receive a strap therethrough that affixes the base plate to a planar surface that prevents movement of the base plate.

46. The system for creating a simulated foot position of claim 26, the calfplate including a heelcup configured to hold the foot in position atop the calfplate.

47. The system for creating a simulated foot position of claim 26, the calfplate being pivotally securable with the base plate, whereby the calfplate moves between a flat position and an elevated position.

48. The system for creating a simulated foot position of claim 47, wherein:
the base plate including at least one swingout moveable between a stowed position and a support position; and
the calfplate including at least one elevator, whereby, when the swingout of the base plate resides in the support position, the elevator of the calfplate engages the swingout thereby locating the calfplate in the elevated position, and, when the swingout of the base plate resides in the stowed position, the elevator of the calfplate bypasses the swingout thereby locating the calfplate in the flat position.

49. The system for creating a simulated foot position of claim 47, the calfplate including a latch whereby, when the calfplate resides in the flat position, the latch engages the base plate thereby locking the calfplate with the base plate.

50. A method for creating a simulated foot position, comprising:
placing a foot atop a base;
moving a footpad pivotally secured with the base to an upright position against the foot such that the footpad maintains the foot in a simulated foot position; and
performing imaging of the foot.

51. The method for creating a simulated foot position of claim 50, wherein placing a foot atop a base, comprising:
moving a calfplate of the base pivotally secured with a base plate of the base to an elevated position; and
placing the foot atop the calfplate.
